(19) United States
(12) Patent Application Publication
(10) Pub. No.: US 2003/0040128 A1
(43) Pub. Date: Feb. 27, 2003

(54) Method of conducting and documenting analyte screening tests

(76) Inventors: James W. Meador, Houston, TX (US); Anne Becknell, Parker, CO (US)

Correspondence Address: TIM L. BURGESS, P.C., 402 OAK LANE, HOUSTON, TX, 77024, US

(21) Appl. No.: 10/228,351

(22) Filed: Aug. 26, 2002

Related U.S. Application Data
(60) Provisional application No. 60/314,759, filed on Aug. 24, 2001.

(51) Int. Cl. G06F 19/00; G01N 33/48; G01N 33/50; G01N 33/543

(52) U.S. Cl. 436/518; 702/19; 713/200

(57) ABSTRACT

A method of conducting and electronically documenting analyte screening testing such as testing for drugs of abuse uses one or more analyte screening media carried in a support, the media expressing results of reaction visibly on the media. The method includes contacting the analyte screening support with a bodily fluid of the donor to allow reaction of the reactant with an analyte tested by the reactant if present in the bodily fluid, then electronically imaging the reacted analyte screening support visibly showing the test results, and incorporating that electronic image into a read-only electronic form. The donor provides an authentication into the electronic form or onto a print copy of the form.

### CROSS REFERENCE TO RELATED APPLICATION

This application is based on provisional patent application No. 06/314,759, filed Aug. 24, 2001, and claims priority therefrom.

### FIELD OF THE INVENTION

The present invention relates to a method for conducting and documenting drugs of abuse or other analyte screening tests using one or more analyte screen media carried in a support.

### BACKGROUND OF THE INVENTION

The increased availability and use of drugs of abuse by the general population has caused employers, governmental agencies (especially those involved in rehabilitation and criminal justice), sports associations and groups, and other organizations, to use drug screening as a condition of employment, to maintain safety in the work place, for parolee monitoring, for criminal prosecutions, and for other valid reasons. Typical drug screening tests are performed for the purpose of quickly identifying on a qualitative basis the presence of drugs in a body fluid, usually urine. If the preliminary screening results are “non-negative”, a complete analysis of the sample may then be carried out in a qualified laboratory. A suitable drug screening device is disclosed in U.S. Pat. No. 5,976,895 assigned to the assignee of this application.

Numerous safeguards are imposed on drugs of abuse or other analyte screening tests to assure validity of results and correct connection of the results to the person being tested. In drugs of abuse testing, it is not unknown for persons being tested to try to cheat the testing by sneaking another person's urine into the testing station or by diluting urine with tap water or by other artifices. This invention is not directed to that problem. This invention is directed to the problem of assuredly preserving and connecting the results obtained to the person tested. The problem of connecting the results obtained to the person tested historically has involved the use of paper custody and control forms purposed to maintain and demonstrate a chain of custody of drug tests in the event the tests are challenged or if the results may become proof in a prosecution involving a criminal act in which the result of the drug test is relevant. A proven chain of custody assures constancy of connection between the urine specimen and the ultimate test result.

A conventional process involves use of a chain of custody paper form supplied by a testing laboratory. Typically the form has peel off accession or blank labels on it. This label usually has a serial number, and may be a bar coded number. A label from the chain of custody form is removed and attached to a sample cup or test container, and the same label is also applied to the support of a separate drug screen media support if that is the manner of test. If the label is blank, a unique identification number such as the donor's social security number is written onto the label.

The test is then conducted by contacting a urine specimen provided in a sample cup or other container with a test medium containing reactants for drugs of interest to the tester. In some instances the test medium is in a well in a container into which urine from the sample cup is decanted or into which the donor voids directly. With a test according to U.S. Pat. No. 5,976,895, the reagents are in media carried on a separate planar support in the form of a card which is placed into the cup.

The test is scored according to the test criteria to identify tests that yielded a negative result and tests that yielded a non-negative result. If the test results are negative, the on-site tester is saved the trouble of completing the chain of custody form but may still have a different form to complete and send to the client-requestor of the test (prospective or existing employer, government agency, etc.) and to the tester's file, reporting the negativity of the test results.

If the test yielded non-negative results, the on-site tester may have the job of preserving the primary test results, as by inserting the labeled, urine-wetted media support in a repository such as a plastic bag that is closed with a tamper proof seal, storing the sealed bag where it cannot be accessed without proper authority, completing another chain of custody form if the storage bag leaves the custody of the tester, and maintaining a database or other record by which the sample can be identified to the chain of custody form and the sample donor (including by accession number) and retrieved from the repository in the event of challenge or other need to refer to the primary record of the test media.

If the test yielded non-negative results, the on-site tester also sends the container housing the urine specimen to a testing laboratory for definitive analysis while keeping a chain of custody intact. The on-site tester completes the principal chain of custody form originated with the test and turns over the chain of custody form to a courier, who signs indicating receipt of the sample and form, and who then transports the sample and form to a testing laboratory, where the form is signed signifying receipt from the courier. The testing laboratory definitively tests a portion of the sample and stores the remainder of it for recovery if there is a challenge to the test results. The definitive test result data are reviewed and scored by the medical review officer for data indicating a non-negative result in excess of any threshold amounts. The medical review officer enters his scoring on the chain of custody form and returns the form to the on-site tester and/or to whomever the officer is otherwise directed to report the results, which may be the clientrequestor.

In many on-site drug screening situations, one person is assigned the tasks of interviewing the sample donor to obtain the needed identification information to complete the necessary form, then provide a sample cup, the receive the sample, then conduct the test, and then score the drug screen test results. In some on-site situations, the person overseeing the test does not want the responsibility of scoring the test knowing that the donor knows his or her identity. Further, privacy considerations give rise to concerns about preserving the anonymity of the person being tested for drugs of abuse. Additionally, when possible it is good methodology to prevent the scorer of the qualitative drug screen test from knowing the identity of the person being scored, to avoid the possibility of bias in test results. Sometimes these concerns can be addressed by having the sample cup provided anonymously to the tester and test scorer, but this involves transfers of the specimen through several hands and introduces greater potential for loss of specimen, mess, testing delay factors, potentials for adulteration between the time of sampling and the time of testing that prophylactically require a tamper evident seal immediately after the specimen is provided, need for additional personnel, and other problems and variables that complicate the testing and may lead to questions about the credibility of the testing results.

U.S. Pat. No. 6,376,251 B1 (the '251 patent) describes electronic transmission of a fax image of a completed paper form from an on-site testing location to a central data management site for recognition and storage of the data on the form. The '251 patent also describes the concept of a reader device, either stand alone or integrated into a computer system, for optically scanning and storing an image of the urine test device and for analyzing the image to determine the result of the test. If the reader device is capable of analyzing the image, it outputs an analysis report (negative or non-negative test results for drugs tested) by indicator lights or textually. A keyboard or other input device connected to the reader allows entry of information identifying and connecting the test subject to the analysis reported by the device. The analysis report may be printed or sent by telephone network or the internet to the central data location. The analysis report from the reader device may also be entered on the paper form that is then faxed to the central data location. The stated purpose of separately sending the analysis report data from the reader device in addition to the faxed image of the completed paper form is to flag faxed image recognition errors and verify that data reported for the same test subject from multiple sources is consistent to assure accuracy. If a faxed image recognition result is flagged, a call is made to the testing site to verify the information.

Since with the system of the '251 patent the possibility exists that a faxed image may have recognition result error which requires verification by the on-site test screening location, the on-site location must retain the urine-wetted media support for purposes of verification or not if the reader device image is not saved after analysis. Thus the '251 patent does not solve the problem of having to bag and seal the urine-wetted media support, store the sealed bag for retrieval, completing another chain of custody form if the storage bag leaves the custody of the tester, and maintaining another database to keep up with the bagged test medium. Nor does the '251 patent provide other benefits of this invention.

### SUMMARY OF THE INVENTION

It is an object of this invention to provide an electronic form for electronic recordation of analyte screening test results and test subject data at an on-site testing location.

It is an object of this invention to provide an electronic form for electronic recordation of analyte screening test results and test subject data at an on-site testing location, for telecommunication of the electronic data not as a faxed image.

It is an object of this invention to provide an electronic form for electronic recordation of analyte screening test results and test subject data at an on-site testing location which can be dispatched by telecommunication to a remote facility for storage without possibility of error from data corruption by image recognition software operating on a faxed image.

It is an object of this invention to provide an electronic form for electronic recordation of analyte screening test results and test subject data at an on-site testing location, for telecommunication of the electronic data to a central database from which purposed reports can be generated without possibility of error from data corruption by image recognition software operating on a faxed image.

It is an object of this invention to provide an electronic form for electronic recordation of analyte screening test results and test subject data at an on-site testing location from which data can be extracted at the on-site location or remotely by telecommunication and uploaded to a database without possibility of error from data corruption by image recognition software operating on a faxed image.

It is an object of this invention to provide an electronic form for authentication by the test donor.

It is an object of this invention to preserve a primary visual record of the result of analyte screening tests in an electronic form for electronic transfer by telecommunication.

It is an object of this invention to preserve a primary visual record of the result of analyte screening tests in an electronic form including analyte screening tests results and test subject data for electronic transfer by telecommunication.

It is an object of this invention to preserve a primary visual record of the result of analyte screening tests in an electronic form for electronic transfer by telecommunication to a referee for scoring of the results of the test.

It is an object of this invention to provide an electronic chain of custody and control form that includes a primary visual record of the result of analyte screening tests in an electronic form.

A method of conducting and electronically documenting analyte screening tests is provided using one or more analyte screening media carried in a support, the media expressing results of reaction visibly on the media. The method includes contacting the analyte screening support with a bodily fluid of the donor to allow reaction of the reactant with an analyte tested by the reactant if present in the bodily fluid, then electronically imaging the reacted analyte screening support visibly showing the test results, and incorporating such electronic image into a read-only electronic form. The form may be a custody control form. The testing, imaging and incorporation of the image into the electronic form may all occur at the on-site testing location. The donor provides an authentication into the electronic form or onto a print copy of the form containing the image of the test results. The authentication into the electronic form or onto a print copy of the form may be a signature, and into the electronic form may be any other form of electronic identification originating from the test subject, such as a voice print, acknowledging the verity of the source of the test results.

The form may be electronically dispatched to a referee for scoring the results of contacting the analyte screening support with the donor bodily fluid as viewed from the image. The referee scores the results of the test, enters the scored results into the electronic form, and electronically dispatches the form containing the scored results to the site of testing. The test site receives the electronic form containing the scored results and has the donor provide an authentication into the electronic form or onto a print copy of the form containing the scored results. If the scoring from the referee indicates a nonnegative result for an analyte, a container containing the bodily fluid of the donor contacted by the analyte screening support, identified according to the identifying information from the analyte screening support label on the image, is accessed and transferred to a testing laboratory for quantitative analysis of its contents.

With incorporation of the primary testing results image on an electronic read-only chain of custody form, a number of benefits accrue. The original urine-wetted test media is subject to fading over time. A color printout of a visual image of a fresh urine-wetted test media is subject to fading over time. A film photograph of the fresh urine-wetted test media is subject to fading over time. However, an electronic image of the fresh original urine-wetted test media on an electronic read-only chain of custody form is preserved without fading over time, so an exact visual record on which a negative or non-negative determination is made is always available on the electronic form. As a read-only image, the image is not subject to alteration. With an exact visual record on which the negative or non-negative determination is made always being available on the electronic read-only chain of custody form, there remains no need to preserve the original urine-wetted test media, or have a separate chain of custody form for storage and retrieval of it or have another record database coupled to the separate chain of custody form to keep up with the identification and location of the stored test media. Mess and paperwork are eliminated, with attendant economic savings.

Further, data in the electronic form that is not the primary visual record of the screening test results does not have to be converted, as by image recognition software, from an image to binary data useable by a digital computer. The data is binary from the beginning. It can be extracted from the electronic form and used in a larger database as desired, while the primary read-only record containing the primary visual record of the screening test results are retained inviolate. Thus, for example, the database source may be used to populate recurring entries in the electronic form automatically on the initial entry of some identifying form. Entry of a name of the tester may populate the remainder of the form with data unique to that tester, saving time and effort in entry of information. Further, reports can be prepared from the database advising on the histories of analyte screening of tested subject and tester, or the number of tests and results conducted at a location, or other reports of interest to the client.

There are numerous other benefits and features of the invention, and these will be described or be apparent to those skilled in this art from the detailed description of the invention taken in connection with the drawings, which are now briefly described.

### DESCRIPTION OF THE DRAWINGS

FIG. 1 depicts a computer usable “Subject Information” template suitable for entry of information and use in accordance with this invention.

FIG. 2 depicts the same template as shown in FIG. 1 with a pull down list adjacent the legend “Reason for Test” shown.

FIG. 3 depicts the “Subject Information” template of FIG. 1 with example entries.

FIG. 4 depicts a computer usable “Exam Observations” with a pull down list adjacent “Opiates” check off boxes shown.

FIG. 5 depicts the same template as shown in FIG. 3 with a pull down list adjacent the “Alcohol” check off boxes shown.

FIG. 6 depicts the same template as shown in FIG. 3 with a pull down list adjacent the “Adulteration Normal” check off box shown.

FIG. 7 depicts the same template as shown in FIG. 3 with a pull down list adjacent the “Adulteration Abnormal” check off box shown.

FIG. 8 depicts a variation of the “Subject Information” template of FIG. 1.

FIG. 9 depicts the same variant template as shown in FIG. 8 with a pull down list adjacent the legend “Reason for Test” shown.

FIG. 10 depicts the same variant template as shown in FIG. 8 with a pull down list adjacent the “via” in the “Send Results to” panel shown.

FIG. 11 depicts a defaults template for settings options.

FIG. 12 depicts the template of FIG. 8 with example entries.

FIG. 13 depicts a variation of the “Exam Observations” template of FIG. 4, with a pull down list adjacent “Opiates” check off boxes shown.

FIG. 14 depicts the same template as shown in FIG. 13 with a pull down list adjacent the “Alcohol” check off boxes shown.

FIG. 15 depicts the same template as shown in FIG. 13 with partial example entries.

FIG. 16 depicts the “Exam Observations” template of FIG. 4 un-scored for test results with other example entries made and showing an acquired image of a drug testing card.

FIG. 17 depicts the “Exam Observations” template of FIG. 4 scored for test results with other example entries made and showing an acquired image of a drug testing card.

FIG. 18 depicts the same template as shown in FIG. 4 with a popup error message shown listing all errors that can be messaged if an error occurs in completion of the form.

FIG. 19 shows a word processor window containing an electronic read-only document file of predetermined layout to which the entered information from the templates of FIGS. 1 and 4 has been exported.

FIG. 20 shows a print out page of the document of FIG. 13.

### DETAILED DESCRIPTION OF THE INVENTION

In accordance with the invention, drugs of abuse screening tests are performed using one or more drug screening media carried in a support, each medium containing a different reactant reactive to a different selected drug of abuse that expresses results of reaction visibly on the medium. Drug screening media carried in a support (herein for brevity sometimes referred to a “drug screening support”) typically are immunoassay strips that express results of reaction on the strip medium. Examples of such strips and use of a planar support for such media are described in U.S. Pat. No. 5,976,896 and are embodied in the “Rapid Drug Screen”® products made by the assignee of this application. Without limitation, other examples of a drug screening support are cups from Roche Diagnostics called “OnTrak Testcup”® which situate test strips for amphetamines, cocaine, morphine, THC, PCP, benzodiazepines and methamphetamines in various combinations in a well in a sample cup as described generally above; the Syva “RapidCup”® from Dade Behring also providing various drugs of abuse tests; and the Medtox Scientific, Inc. “Profile-II”™ and “Verdict-II”™ test systems. In these systems, typically, when no drug is present in the urine sample, antibody-colloidal gold complexes in solution migrate along a strip medium and bind to an appropriate conjugate on the medium at a labeled location indicated on the support for the medium; where they bind, a red line is generated. When a drug tested for is present in the urine sample near or above a cutoff concentration, the antibody-colloidal gold binds to the drug before it migrates along the strip media. As a result there is no binding of the antibody-colloidal gold with the conjugate at the drug-specific location marked on the support, and consequently no red line appears. A control or validation line is typically present to show the antibody-colloidal gold is present. Therefore, if two red lines are present (validation line and test line), the test is negative; if only the base validation line is present, the test is non-negative.

While the foregoing explanation is an example of an analyte screening test producing a visible result on a testing support, the illustration is non-limiting, and other testing systems that produce a visible indication of test results are within the scope of this invention.

In an embodiment of the invention, the test is conducted and documented at the on-site testing location by applying an identification of the donor to the drug screening support, and upon receipt of a bodily fluid sample from the donor, contacting the drug screening support with the bodily fluid of the donor to allow reaction of the test reactant with a drug tested by the reactant if present in the bodily fluid. After the test is completed, the invention includes electronically imaging at the on-site testing location the reacted drug screening support visibly showing the test results.

Acquiring an electronic image of the bodily fluid-contacted-and-identification-annotated drug screening support visibly showing the results of contacting the drug screening support with the donor fluid may be done in a number of ways.

If the bodily fluid-contacted-and-identification-annotated drug screening support is generally planar so that it may be scanned on a flat bed scanner (which has limited depth of field), suitably the image may be acquired with such a scanner electrically connected to a computer. The bodily fluid-contacted-and-identification-annotated drug screening support showing the results of contacting the drug screening support with the donor bodily fluid is suitably placed in an optically clear envelope and the envelope is placed on the scanner in a location controlled by a position template.

Alternatively, and especially where the test support has a significant depth, it is advantageous to use a charge coupled device (“CCD”) for acquiring an image of the test result. Unlike the very small depth of field available with a scanner, the use of a CCD for acquiring the image of the drug screening support allows use of optics that can be optimized for a particular non-planar form. An electronic box as hereinafter described is designed to use a CCD for image acquisition.

The acquired image is stored in a medium from which the image is electronically retrievable. In an embodiment, the image capturing device suitably makes use of the color of the test line. Tests in which the validation and test line are red will absorb blue light at the validation and test lines. Consequently, illumination of the test card with blue LED's (light emitting diodes) allows the use of a relatively inexpensive black and white CCD.

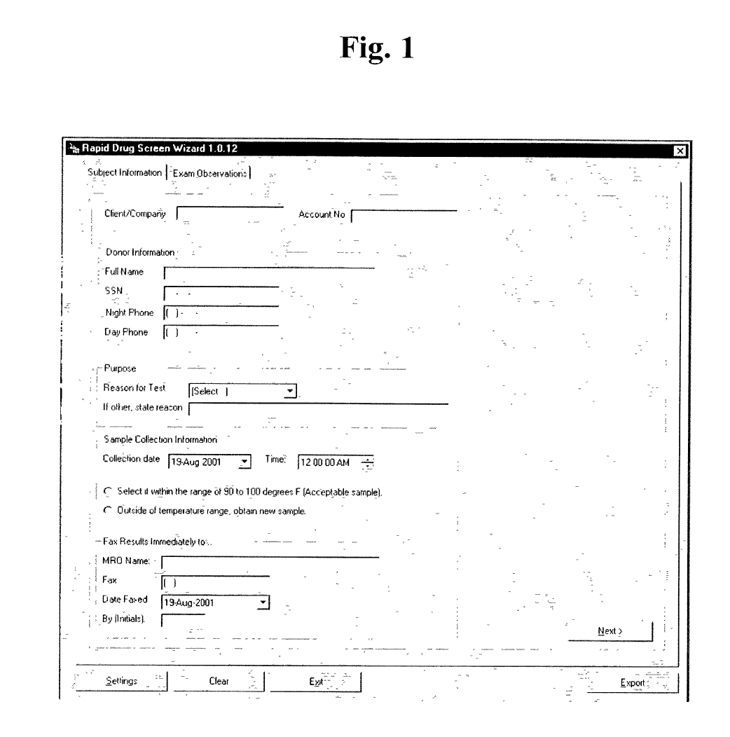

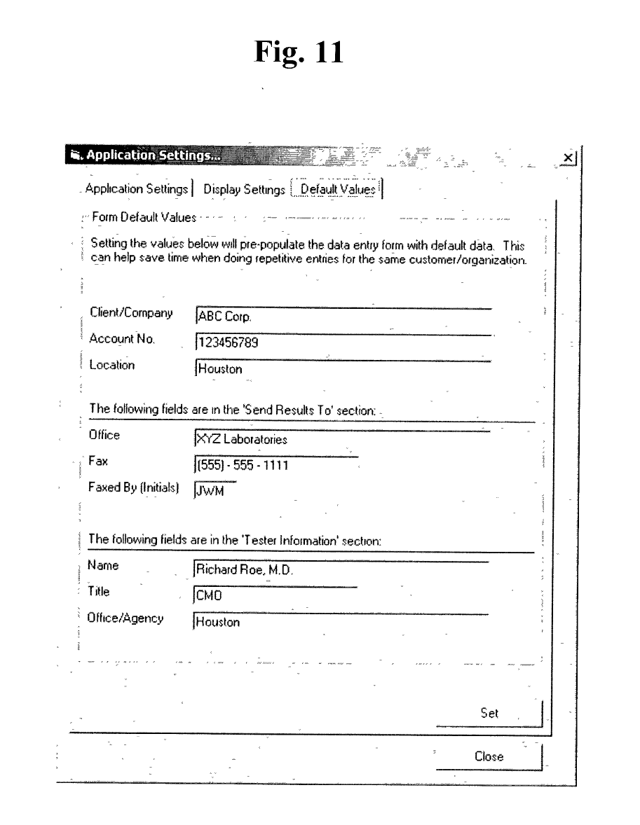

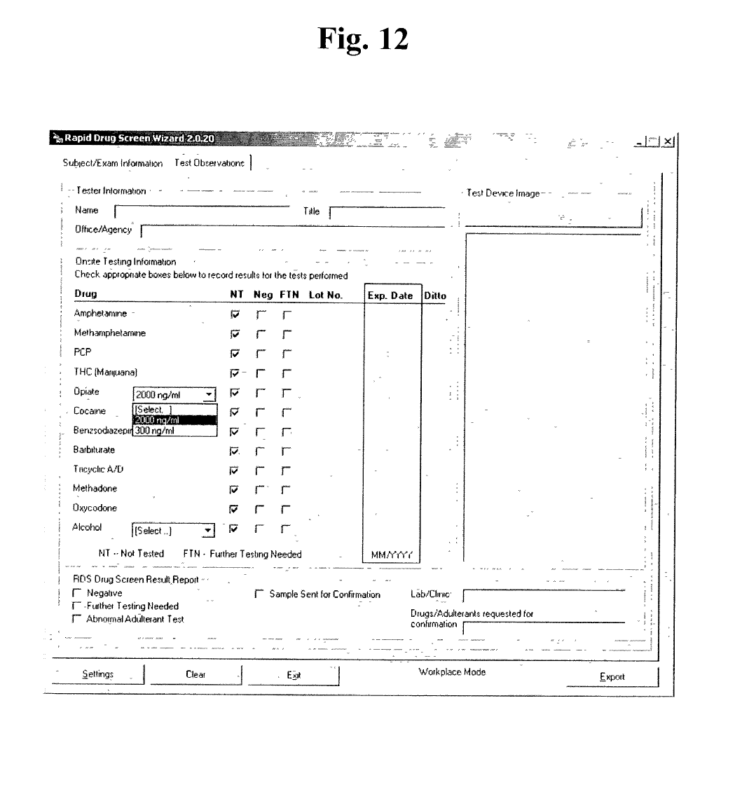

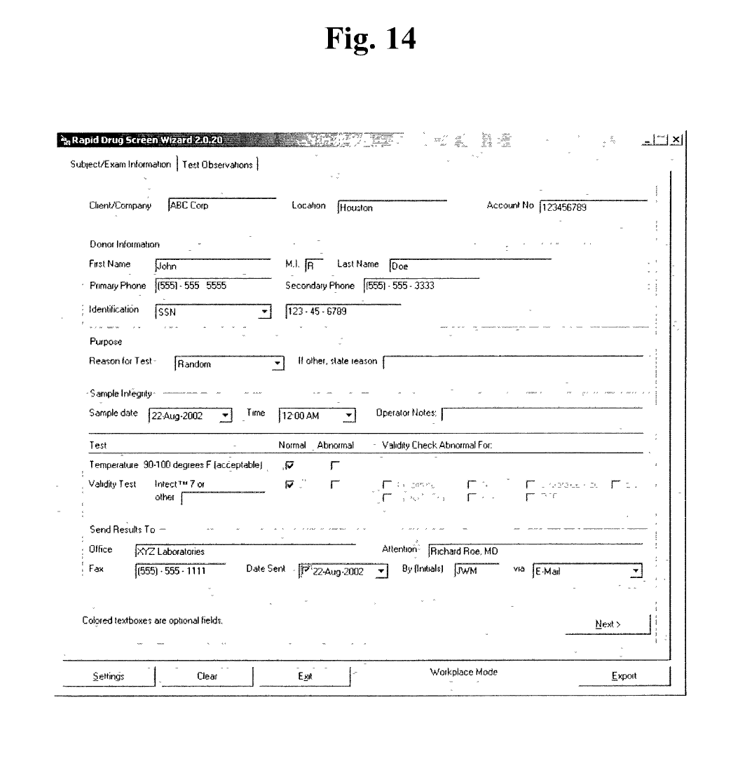

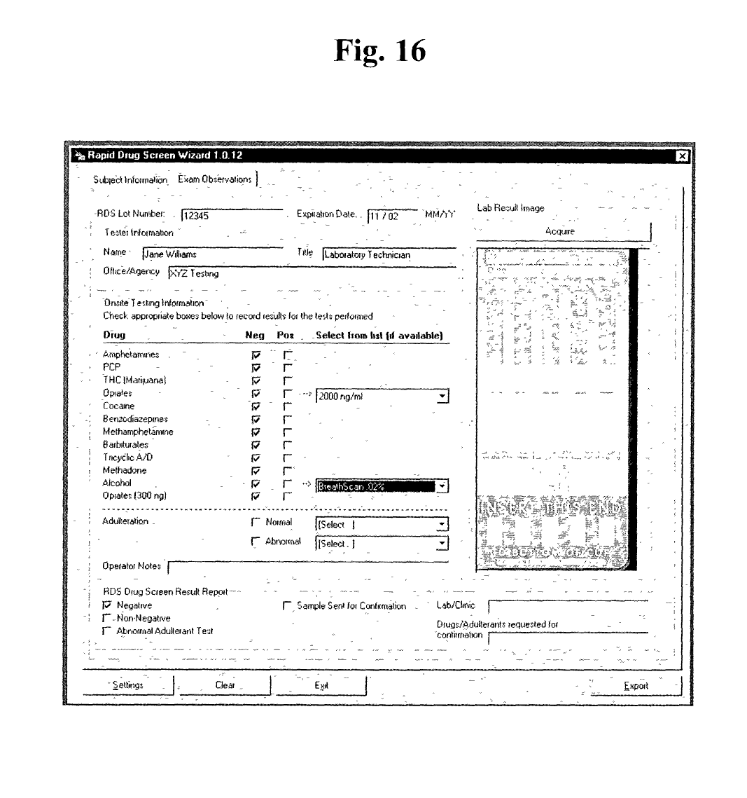

After the image is acquired and stored, the electronic image is incorporated into a read-only electronic form at the site of testing. For example, software provided to a user places an icon on the user's computer desktop. Double clicking on the icon brings up a template which instructs the user how to fill out the template form and forces them to fill out all of required entries on the form. The user places a reacted drug screening support, for example a planar drug screening card, on a flat bed scanner, in a template so the software knows the position of the drug screening report on the scanner. The user clicks an “acquire“button on the template and the scanner automatically scans the drug screening support and acquires the scanned image. FIGS. 1-16 depict electronic template forms for entry of information and inclusion of an acquired image of the test results used in accordance with this invention. After the user has filled out the templates completely, the information on the templates including the acquired image of the drug screening card is exported into a read only word processor file in a predetermined arrangement or format of information. FIG. 17 depicts an electronic document created from entries in the electronic forms. A printer prints out the word processor document and automatically saves it in read-out form with identifying information such as the donor's social security number followed by the date. It is saved suitably as the file type of the word processor or alternatively as an image file (TIFF, JPEG or other suitable format) or in an Adobe® portable file document (*.pdf®) format. FIG. 12 shows a printout of the electronic document, ready for signature by the tester and the test subject. The donor and the operator sign the form and the form can be filed and copies distributed as protocol provides. The electronic document can be e-mailed as a read only attachment.

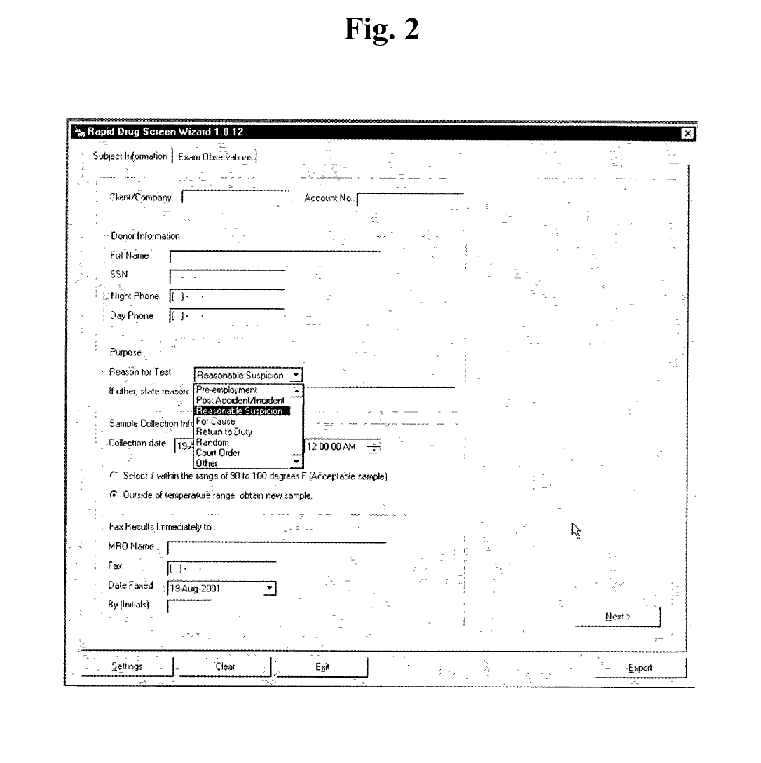

The foregoing is described more particularly in reference to alternative templates shown in FIGS. 1-20. Referring to FIG. 1, an electronic form template is illustrated accessible by two tabs. One tab is labeled “Subject Information”; the other tab is labeled “Exam Observations”. The template accessed by the tab labeled “Subject Information” includes entry box for the name of the “Client/Company” requesting a drug screening test and the “account No.” of the Client/Company. In a grouping under the legend “Donor Information” are boxes for entry of the “Full Name” of the donor of the bodily fluid (normally urine) to be tested for presence of drugs of abuse, the “SSN” (social security number) of the donor, the “Night Phone” and “Day Phone” where the donor may be reached. Under the group legend “Purpose” is a pull down list box “Reason for Test” accessed by clicking onto the down arrow. The pull down list of the “Reason for Test” box is shown in FIG. 2., showing as reasons “Pre-employment”, “Post Accident/Incident”, “Reasonable Suspicion”, For Cause”, “Return to Duty”, “Random”, “Court Order” and “Other”. An additional box legended “If other, state reason” referring to the “Other” if that was clicked for the “Reason for Test”.

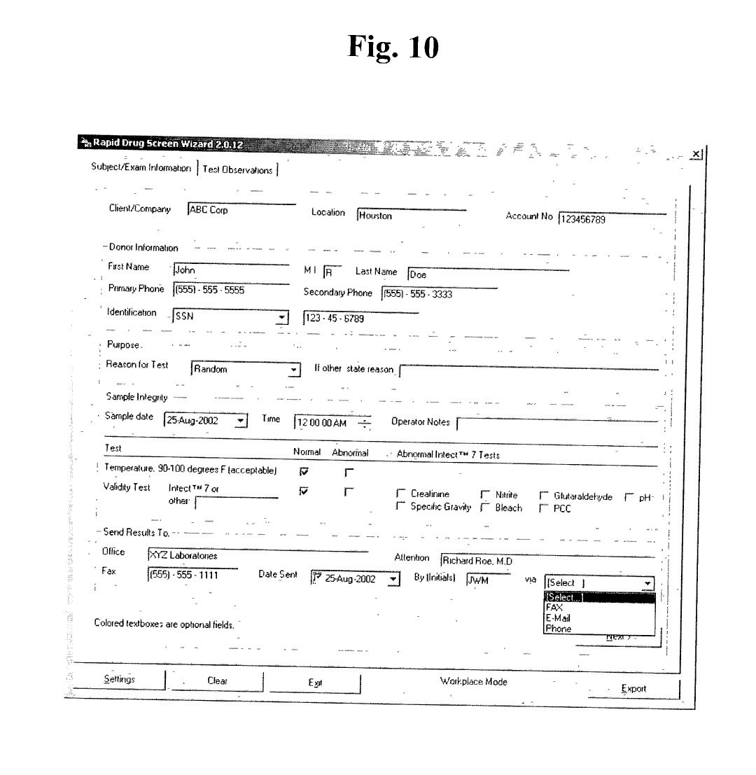

Continuing as respects FIG. 1, grouped under “Sample Collection Information” are two boxes in which information is automatically supplied by the computer, “Collection Date” and “Time”. Below these automatic entry boxes are select buttons labeled “Select if within the range of 90 to 100 degrees F. (Acceptable sample)” and “Outside of temperature range, obtain new sample.” A temperature sensitive indicator is affixed to the container, which tell-tales whether the sample is at the correct temperature. Finally, under the group legended “Fax Results Immediately to . . . ” are boxes labeled “MRO Name” for entry of a medical review officer's name who is to receive results information contained in an export document file as shown in FIG. 10 and described later, “Fax” for the MRO's fax number, Date faxed (supplied by the computer) and “By (initials”) for the initials of the person responsible for faxing the document.

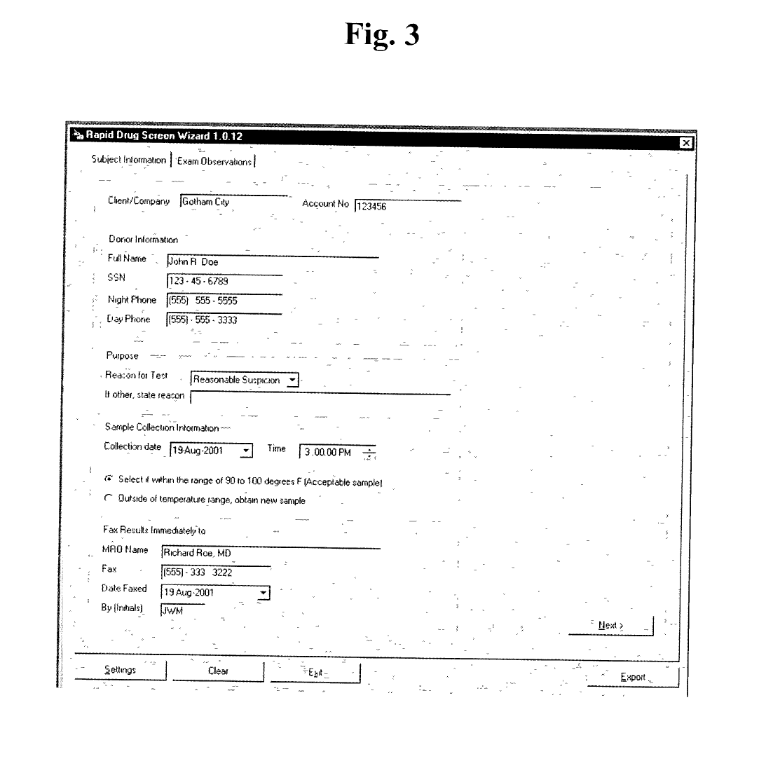

Referring to FIG. 3, an example of the template of FIG. 1 is shown with information completed in the appropriate boxes.

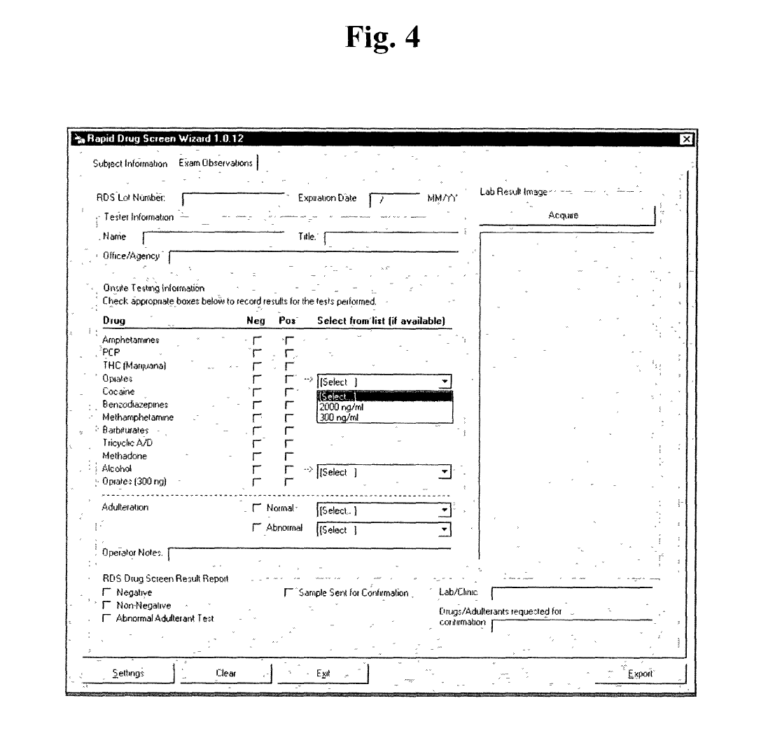

Either by clicking the “Next” button or by clicking onto the tab labeled “Exam Observations”, the “Exam Observations” template is presented on the computer screening. This template (refer to FIGS. 4-7) contains boxes for entry of information concerning the “RDS Lot Number” and Expiration Date in MM/YY format. “RDS” is an abbreviation for “Rapid Drug Screen”®, and in a comparable template would be replaced by a generic or proprietary name of another drug screening system. The purpose of entry of the information is to assure that the test is not conducted using a drug screening system whose expiration date has expired.

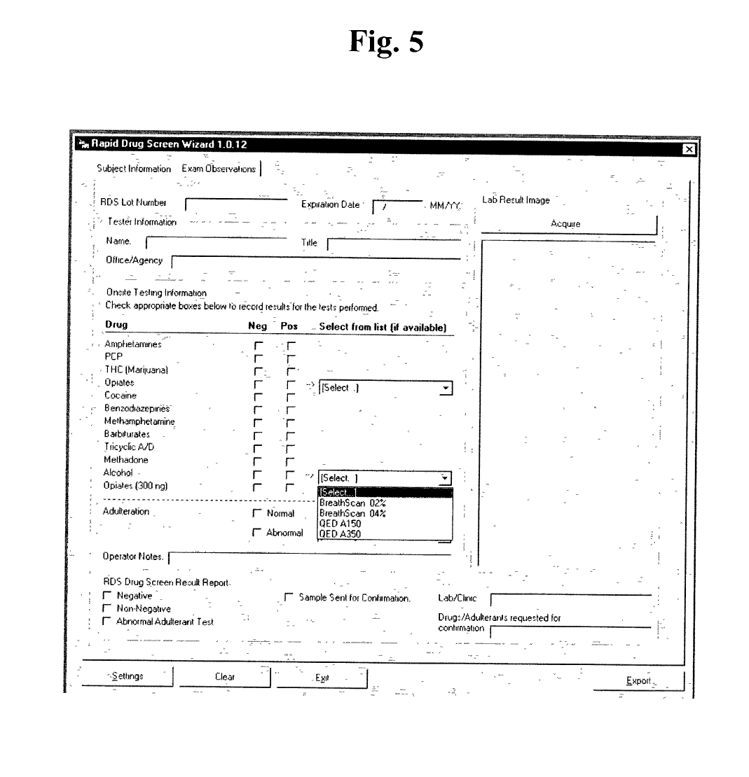

Below the lot and expiration date boxes, in a “Tester Information” grouping, are boxes for entry of the “Name”, “Title” and “Office/Agency” of the person who conducts the test using the drug screening system employed. Below these boxes are parallel vertical columns of boxes, one column labeled “Neg” (for negative) and one column labeled “Pos” for non-negative (positive) test results. Adjacent each box are examples of names of drugs that may be tested in a screening. Not all drugs are tested in one screening. For example, the assignee of this application produces commercial drug screening cards in two 2 panel configurations testing for cocaine and marijuana in one and methamphetamines and marijuana in another; in two 5 panel screens, one for cocaine, opiates, marijuana, PCP and amphetamines, and the other for cocaine, opiates, marijuana, methamphetamines, and amphetamines; in an 8 panel screening, for cocaine, opiates, marijuana, PCP, methamphetamines, amphetamines, barbiturates, and benzodiazepines; and in a 9 panel screening for all the drugs of the 8 panel screening, plus for tricyclic antidepressants. Other tests included on the template are tests for “alcohol” and methadone. As shown in FIGS. 4 and 5 there are pull down lists for differing threshold levels at which opiates and alcohol are tested.

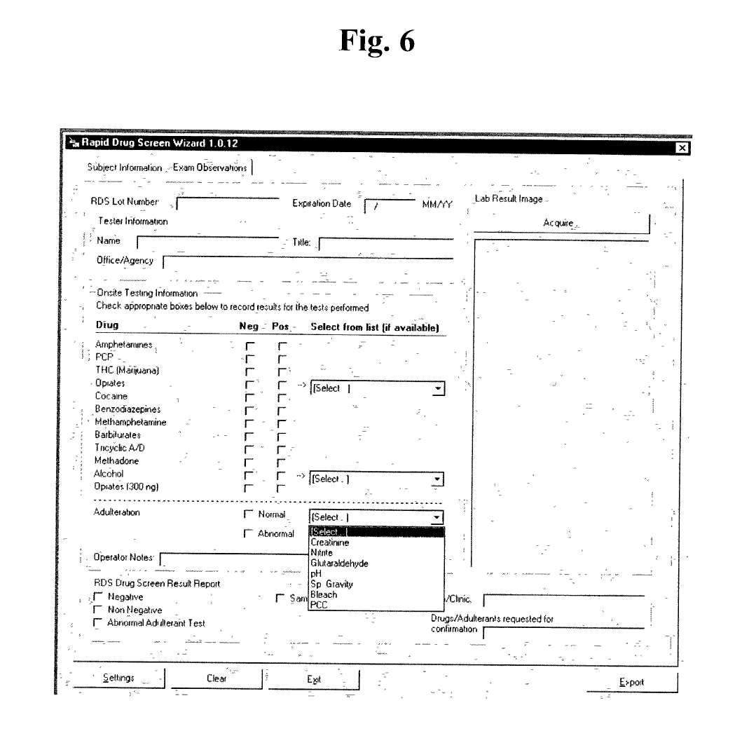

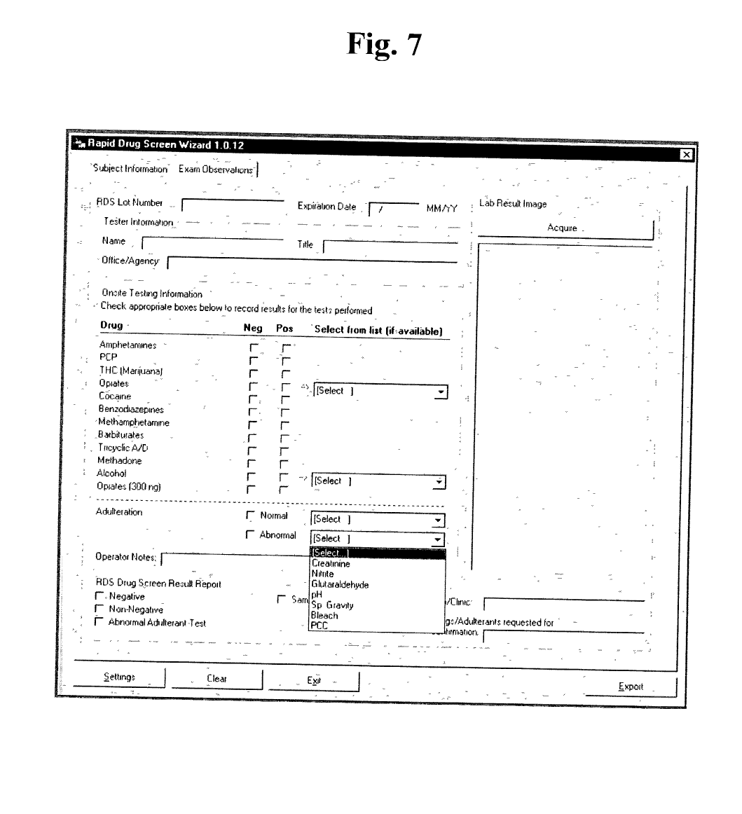

Below the drug grouping, are boxes labeled “Normal” and “Abnormal” in an “Adulteration” grouping. Each of the “Normal” and “Abnormal” boxes has a pull down list with listings that may be selected (see FIGS. 6 and 7). A box adjacent “Operator Notes” allows free form entry. Below the “Operator Notes” box is a grouping under “RDS Drug Screen Result Report” containing check boxes adjacent “Negative”, Non-Negative”, “Abnormal Adulterant Test” “Sample Sent for Confirmation” and information entry boxes adjacent “Lab/Clinic” and “Drugs/Adulterants requested for confirmation.”

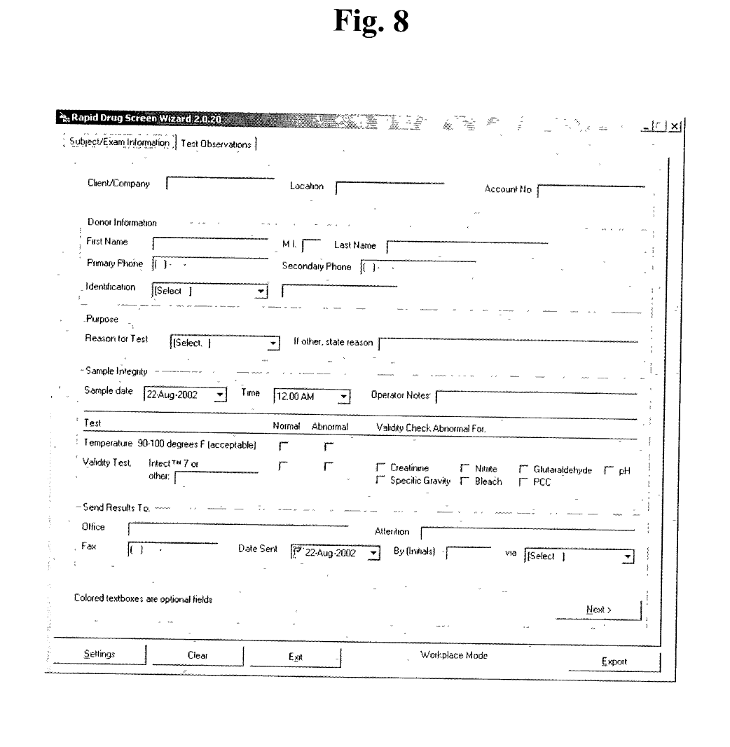

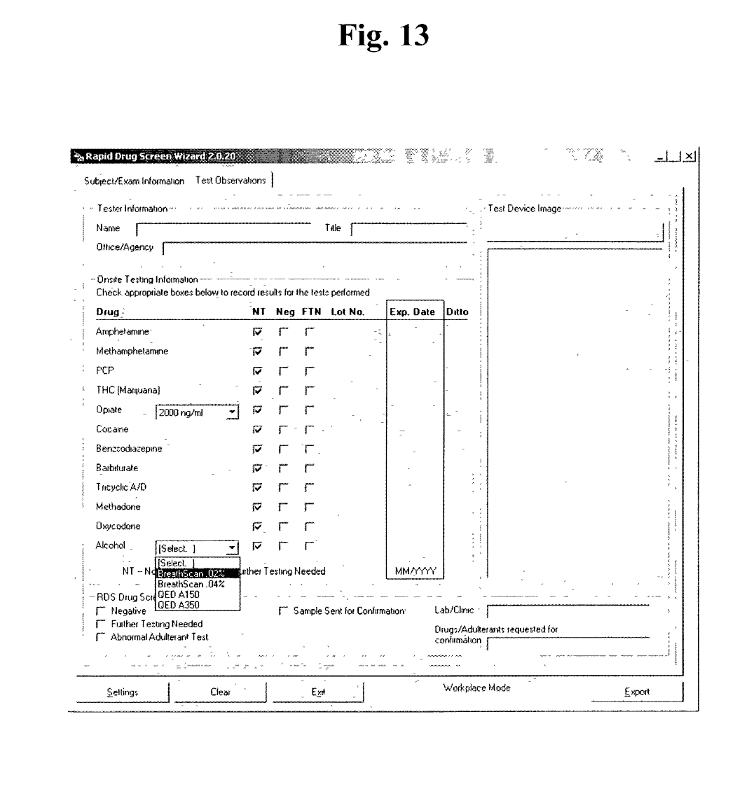

FIGS. 8-11 show a variation of the “Subject Information” template depicted in FIGS. 1-3. FIGS. 13-20 show a variation of the “Exam Observations” template depicted in FIGS. 4-7. In the variant template shown in FIGS. 8-11, the template tab is renamed “Subject/Exam Information” to reflect in part movement and reformatting of the adulteration panel entries from the “Exam Observations” template of FIGS. 4-7 to the “Subject/Exam Information” template of FIGS. 8-11. The adulteration panel is renamed “Validity Test” with adulterants shown in a check box instead of a pull-down list. A panel is provided for identification of the test used to validate that the urine specimen is not adulterated and results of the tests. Tests such as Intect 7™ test strips or other tests detect levels of creatinine, nitrite, glutaraldehyde, pH, specific gravity, bleach, and pyridinium chlorochromate (“PCC”). Screening for creatinine, pH and specific gravity aids in the detection of common attempts to defeat urine drug screens such as dilution or adulteration of the specimen. Screening for nitrite, glutaraldehyde and bleach reveal attempts to foil drug screens by interfering with the reagents or result interpretation components of the drug screening device. The presence of pyridinium chlorochromate in the urine is indicative of adulteration since it is not a normal constituent of urine. A positive result reading may also indicate the presence of other oxidative adulterants.

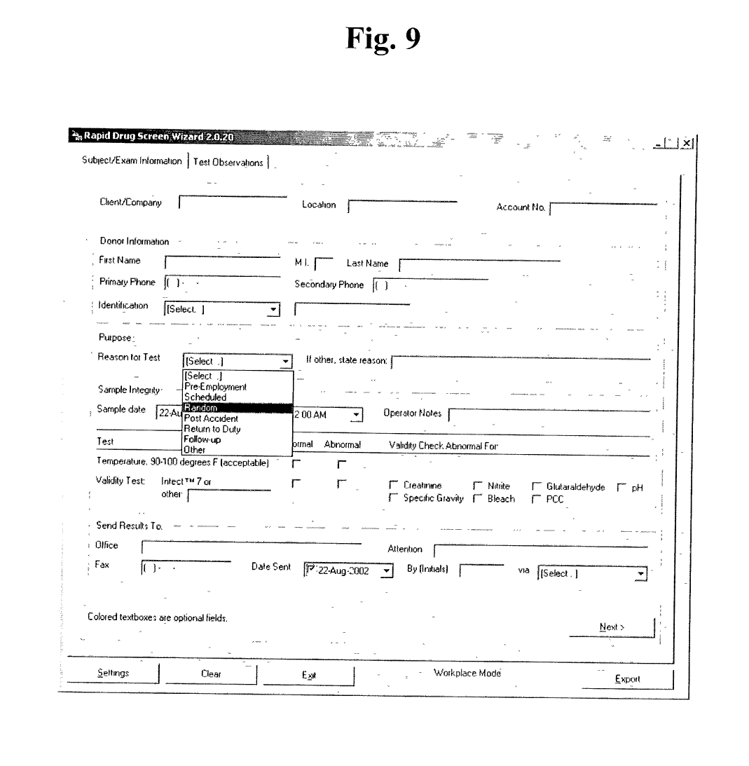

Other changes from the template of FIGS. 1-3 are an information entry box to identify location of the client, a plurality of information entry text-boxes instead of one to allow segmentation of the donor's name into first and last name fields, a plurality of information entry text-boxes instead of one to allow naming the office to which results are to be sent and a name at the office to whom the results are to be directed, instead of naming only a MRO (medical review officer), and re-labeling with some consolidation of some information entry text-boxes and labels in pull down lists.. Certain textboxes are colored to indicate that the information is optional (absence of the information will not trigger an error message) or may be entered as a default value, as explained below. The additional text-boxes allow fields for entry of additional information that can be datamined to produce special reports for the client. FIG. 9 shows a pull down list (compare to FIG. 2) as does FIG. 10, in the latter, for methods for sending the results that include use of e-mail.

A test setting button on either template “Subject/Exam Information” or “Test Observations” accesses application settings templates that include “Default Values” text-box for entry of default values for repeating clients, to testing laboratories for which non-negative or “further testing needed” urine specimens are to be sent, as well as for the tester. The ability to enter default values save entering the same information each time a report is prepared as applicable. FIG. 12 shows a completed template of FIG. 8.

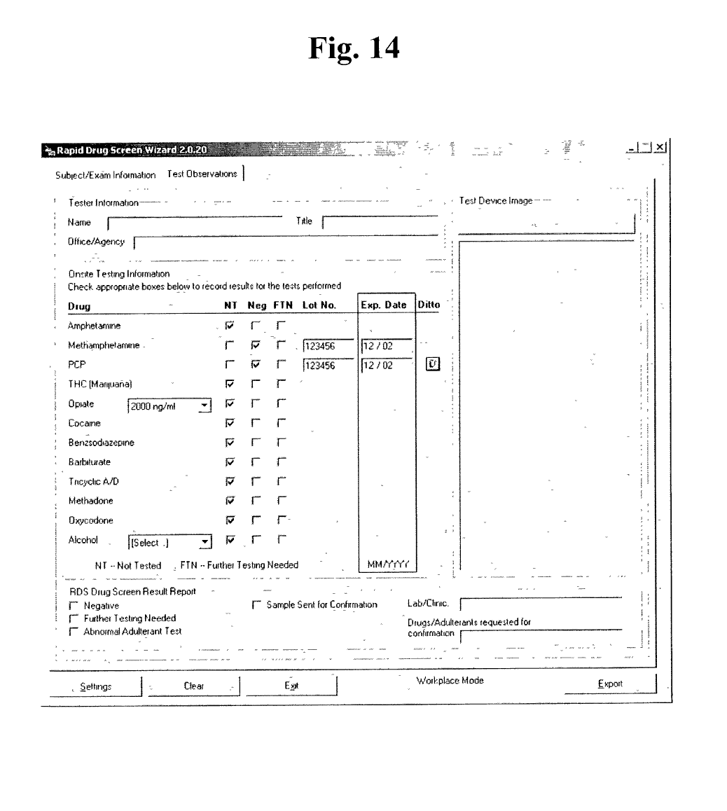

Referring to FIGS. 12-15 that show a variation of the “Exam Observations” template depicted in FIGS. 4-7, the template tab is renamed “Test Observations”. Principal changes from the template shown in FIGS. 4-7 are absence of the adulteration panel moved to the “Subject/Exam Information” template, relocation and expansion of the RDS Lot No./Expiration Date panel to a location in apposition to each drug for which a test is made, addition of a new NT (“Not Tested”) check box field, and re-labeling some fields (e.g., “Pos” to “FTN” for further testing needed). FIG. 12 shows continued use of a pull down list (compare to FIG. 5). FIG. 15 shows the “Test Observations” template partially filled in, showing two drugs were tested (methamphetamine and PCP) and found negative. Entry of a test result forces opening of a text-box which must be completed to show lot number and expiration date of the test. A “ditto” icon allows convenient entry of the lot number identification and expiration if a multi-test panel was run from one test card.

All of the foregoing may be done before the image is acquired. The image is acquired as described above. Acquisition of the image into the electronic form template is depicted with respect to the “Exam Observations” template of FIGS. 3-7. The same process will be understood accomplished with the template “Test Observations” template of FIGS. 12-15 without need of further depiction.

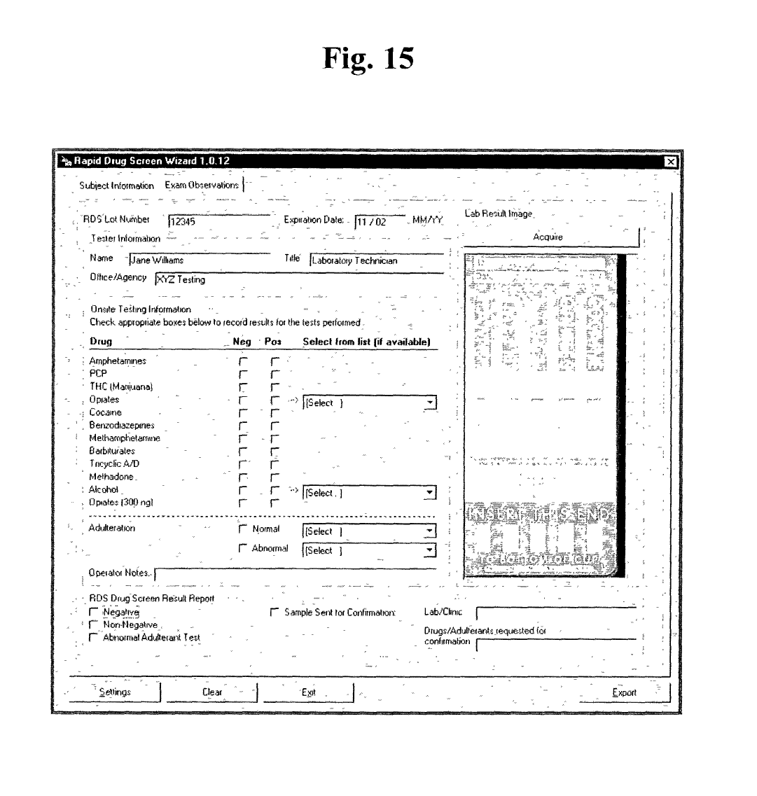

Referring to the computer forms template of FIG. 7 or FIG. 16, either before or after entering the data into the template of FIG. 7, the button labeled “Acquire” is clicked with a mouse. This actuates the flat bed scanner or the CCD box which captures the drug screening support image and the computer stores the image in a medium from which the image is electronically retrievable. Alternatively, the image may be imported into the computer using a memory card or a USB or other connection as explained below. At the same time the image is transferred into a provided panel in the electronic form template for “Exam Observations” as shown in FIG. 16. The information for entry into the “Neg” or “Pos” columns may be read directly from the urine-contacted, signed and labeled drug screening support before its is scanned or the information may be entered after the image is acquired and transferred into the electronic template. If the computer includes software for analyzing the test card results, the software may enter the results in the appropriated “Neg” or “Pos” columns. In FIG. 16, the drug screening support in the example tests for “AMP” (amphetamines), “METH” (methamphetamines), “THC” (marijuana), “OPIATES”, and “COCAINE”. The tests are negative and are so entered in the template (see FIG. 17). The label in this example is hand entered with a social security number of the donor below the placed for signature of the donor, shown here signed by the donor (“John Doe”).

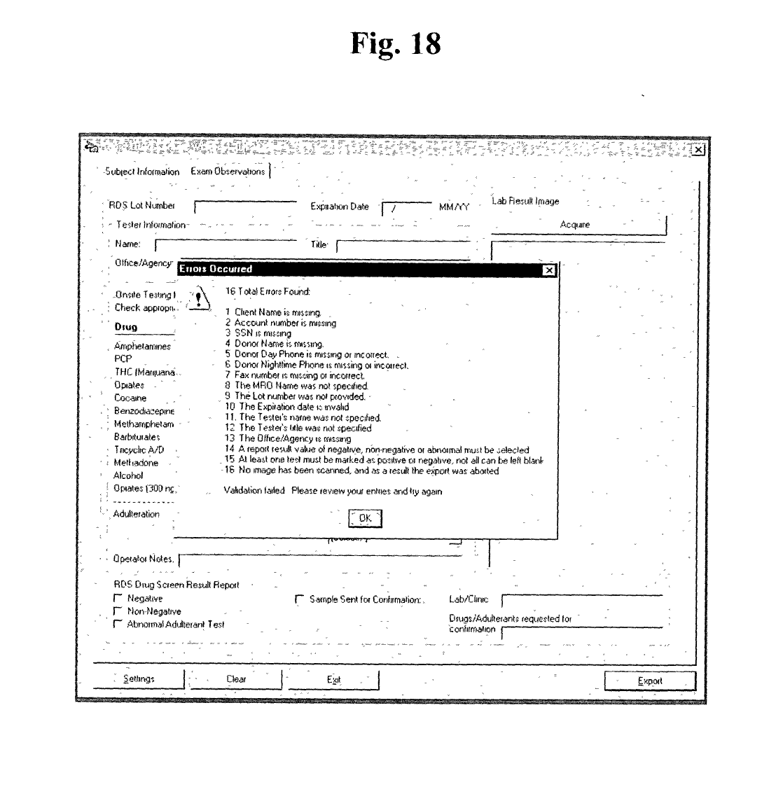

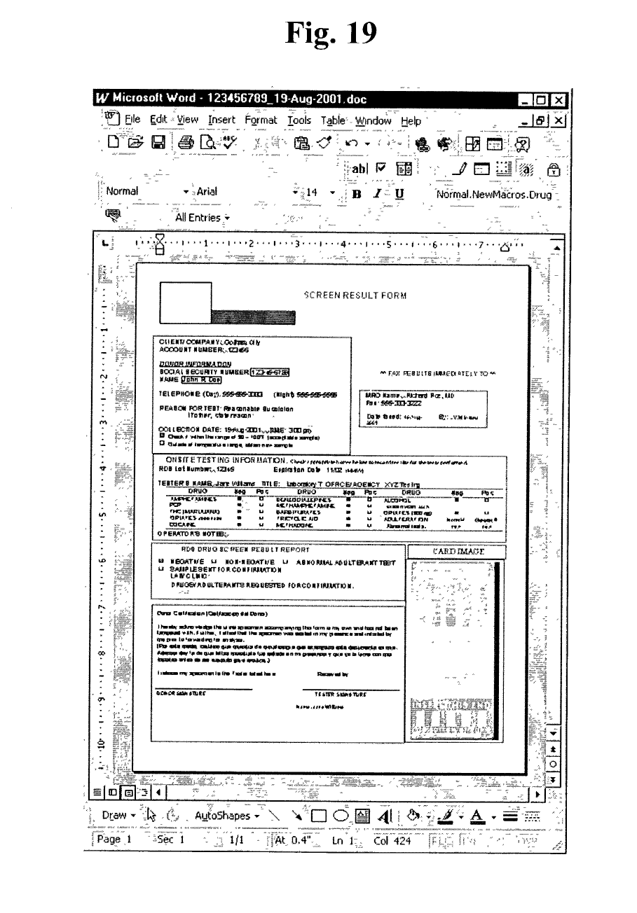

Once the template is filled with the relevant data and the image of the urine-contacted, signed and labeled drug screening support is acquired and transferred into the template, the operator clicks on the “Export” button, seen at the lower right hand corner of the template. If any box is not completed or checked that must be completed for the export function to occur, a message will pop up stating one or more of the errors of completion. FIG. 18 shows the full list of error messages that can pop up, depending on the boxes not completed or checked. If all entries that must be made are made, then the export function will proceed and all the entered information, including the image of the drug screening support, will be exported from the two electronic templates into an electronic read-only document file of predetermined layout, as depicted in FIG. 19. In the example depicted in FIG. 19, the data is exported into predefined fields in a Microsoft Word® word-processor. Preferably, and as shown in FIG. 19, the exporting procedure includes naming the electronic read-only document file by a convention drawing from unique donor information sourced from an entry in said template. In the example, the unique donor information sourced from an entry in said template is the donor's social security number and the date of the test (see FIG. 3 and see in FIG. 19 the title bar of the Word® window showing the name of the electronic read-only document file (“123456789_{—}19 Aug 2001.doc”).

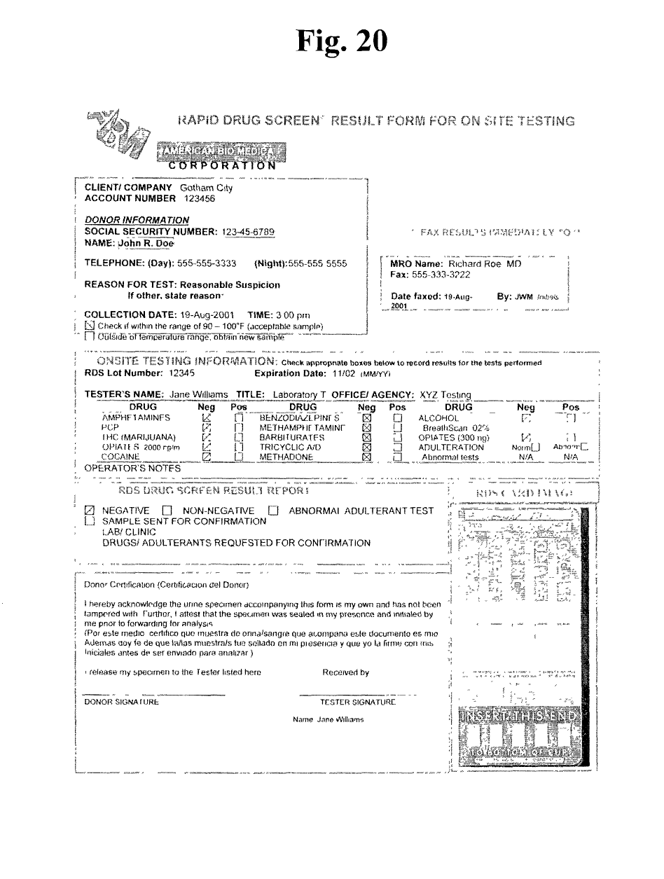

The electronic read-only document file is printed to produce a hard copy that the donor and on-site tester can sign, illustrated in FIG. 20.

In an alternative embodiment, the flat bed scanner is replaced with a small transportable box. The on-site or in-the-field operator inserts into the box the drug screening support (test card), which has a barcode or other identification label placed on it. The card is illuminated in the box with blue LED's (light emitting diodes). The test indicators on the test card are red, and they will absorb blue light. Using a black and white CCD, a black and white image of the card is acquired with the red test strips optimized optically. The barcode or other label eliminates needing a keyboard to be attached to the box to identify the donor. The image is processed and stored on a detachable flash memory or other data storage device that can be coupled to a computer to retrieve the image. Instead of blue LED's a white light source can be used and a RGB CCD or a CMYK CCD can be used to capture color images.

The box can be used in the field and a memory card or other removable recording medium from the box may be plugged into a computer later. Or, the box may be coupled directly to a computer as by a USB, firewire, SCSI, parallel, serial or other port. In any of these embodiments the computer acquires the image from box memory, and incorporates the image into a read-only electronic form. If the on-site tester is not to score the test results, this optionally allows the image to be e-mailed to a referee. The referee can read the image of the test card and score it as negative or non-negative, and then can e-mail the score to the on-site tester. If the result is non-negative the operator forwards the sample to a testing laboratory for confirmatory analysis with a print out of the electronic document signed by the tester and the test subject.

As respects annotating the drug screening support, an approach within the invention is to affix an accession label or blank label (from a form) which can be supplied with an anonymous but unique code to the drug screening support as well as to the sample cup (or in the case of the stored and then printed electronic form—take three identical labels and put one on the printed form, one on the cup and another on the drug screening support). The card is then imaged (scanner, CCD or other). All the information needed to track the test subject urine donor and the form is on the drug screening support image. If it is not wanted to apprise a person scoring the test results of the identity of the test subject, the drug screening support image alone (with label) is e-mailed to the referee, who scores it. The referee then replies to the e-mail saying basically, “It's non-negative send the sample to the lab” or “It's negative don't forward anything.” The image is preserved on the “replied” e-mail with the score. The tracking is done by the label which numerically couples the drug screening support to the form (hard copy or electronic) and then the donor. Upon receipt of the e-mail back from the referee which contains the original image and the score from the referee, the operator brings up the form by sequence number as located on the image (in this case the form will be filed by sequence number instead of social security number-date and can be done manually or by barcode) and makes the appropriate entries on the form and saves/files it. If non-negative the bodily fluid sample is accessed by the same number and forwarded onto the lab.

Instead of printing a completed form containing an image of the test result to have the donor sign the form, an electronic signature may be captured from a pressure sensitive pad and the signatures of the tester and the test subject electronically memorialized on the completed electronic form containing test result image. Another manner of authenticating the test subjects consent to the veracity of the data and imaged test result can be by a digital voice consent or other suitable digital imprint signifying consent recorded and placed in the electronic form.

Alternative to the embodiment described above in which data is input in on screen templates and exported into another form for printing or e-mailing, an electronic form can be provided with fill-in fields that when saved to file can be saved in read-only form, and the electronic file itself can be printed in the format of the fill-in form or can be electronically transmitted as by e-mail. Alternatively, the form with included image can be posted to a server and accessed by a remote referee or any other authorized person having clearance to see the completed form and view the image.

The scope of the invention is set forth in numbered claims below. It will be understood that this invention is susceptible to modification in order to adapt it to different usages and conditions, and accordingly, it is desired to comprehend such modifications within this invention as may fall within the scope of the invention as now set forth.

We claim:

1. A method of conducting and documenting an analyte screening test using one or more analyte screening media carried in a support, a medium in said support containing a reactant reactive to a selected analyte that expresses results of reaction visibly on said medium, comprising:
a. contacting said analyte screening support with a bodily fluid of the donor to allow reaction of a said reactant with an analyte tested by the reactant if present in the bodily fluid;
b. electronically imaging the reacted analyte screening support, said reacted support showing the results of contacting the analyte screening support with the donor bodily fluid; and
c. incorporating the electronic image into an electronic form, said form containing places where information concerning the donor is entered.

2. The method of claim 1 further comprising having the donor provide an authentication into said electronic form or onto a print copy of the form.

3. The method of claim 2 in which the authentication is a signature.

4. The method of claim 1, further comprising scoring the results of the test at said site of testing and entering the results of said scoring into the electronic form.

5. The method of claim 1, further comprising electronically dispatching the form to a referee for scoring the results of contacting the analyte screening support with the donor bodily fluid as viewed from said image.

6. The method of claim 5 further comprising said referee scoring the results of said test and entering the scored results into said electronic form.

7. The method of claim 6 further comprising electronically dispatching said form containing said scored results to said site of testing.

8. The method of concept 7 further comprising receiving containing said scored results at said testing site and having the donor provide an authentication into said electronic form or onto a print copy of the form containing said scored results.

9. The method of concept 6 further comprising if the scoring from the referee indicates a non-negative result for an analyte, identifying a container containing the bodily fluid of the donor contacted by the analyte screening support according to the identifying information from the analyte screening support label on the image, and accessing said container for quantitative analysis of its contents.

10. The method of concept 1 further comprising having the donor sign the analyte screening test support before imaging the support.

11. The method of claim 1 further comprising applying a machine readable code onto said analyte screening report before imaging the support.

12. The method of claim 11 in which said same machine readable code is applied to a container containing said bodily fluid.

13. The method of claim 12 further comprising entering on said electronic form information including donor identification information;

14. The method of concept 13 in which said electronic form comprises a template, and further comprising, exporting the entered information from said template into an electronic read-only document file of predetermined layout.

15. The method of concept 14 further comprising providing said document file electronically to a requester of the analyte screening test conducted on the bodily fluid of the donor.

16. The method of claim 1 in which said analyte is a drug of abuse.

17. The method of claim 1 further comprising telecommunicating said electronic form to a data facility for storage of the information on said form.

18. The method of claim 17 further comprising extracting information from said form for use in a report.

19. The method of claim 18 in which said report includes information extracted from other such forms.

20. A method of conducting and documenting drug of abuse screening tests using one or more drug screening media carried in a support, each medium containing a different reactant reactive to a different selected drug of abuse that expresses results of reaction visibly on said medium, comprising:
a. applying an identification label to a said drug screening support and a urine sample container;
b. receiving a sample container containing urine donated by a donor, said sample container having said applied label;
c. contacting said drug screening support with the urine of the donor to allow reaction of a said reactant with a drug tested by the reactant if present in the urine;
d. having the urine donor sign said drug screening support including said label;
e. electronically imaging the reacted and labeled drug screening support at the site of testing by said drug screening support, said reacted support showing the results of contacting the drug screening support with the donor urine; and
f. incorporating the electronic image into an electronic form at the site of testing, said form containing places where information concerning the donor is entered.
